(19) United States
(12) Patent Application Publication
(10) Pub. No.: US 2005/0020608 A1
(43) Pub. Date: Jan. 27, 2005

(54) Crystalline cetirizine monohydrochloride

(75) Inventors: Manne Satyanarayana Reddy, Hyderabad (IN); Srinivasan Thirumalai Rajan, Hyderabad (IN); Uppala Venkata Bhaskara Rao, Hyderabad (IN); Konda Srinivasa Reddy, Hyderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/809,192

(22) Filed: Mar. 25, 2004

(30) Foreign Application Priority Data
Mar. 25, 2003 (IN) 252/MAS/2003

(51) Int. Cl. A61K 31/495; C07D 241/04

(52) U.S. Cl. 514/255.04; 544/396

(57) ABSTRACT

A novel crystalline form of cetirizine monohydrochloride and processes for making the crystalline form as well as compositions, pharmaceutical compositions, and methods utilizing the crystalline form are described.

### CROSS-REFERENCE TO RELATED APPLICATION

This application claims priority of Indian Patent Application No. 252/MAS/2003, filed Mar. 25, 2003, of which entire content is incorporated by reference.

### BACKGROUND OF THE INVENTION

Cetirizine is an orally active, long-acting histamine H₁ receptor antagonist. It belongs to the second generation of H₁ histamine receptor antagonists that are thought to offer some significant advantages over the first generation compounds. The advantages are believed to include less sedation, low anticholinergic activity, and longer acting duration with the resulting improves patient compliance. Cetirizine is used for the treatment of allergic syndromes, such as chronic and acute allergic rhinitis including seasonal and perennial allergic rhinits, allergic conjunctivitis, pruritus, urticaria, and the like.

### SUMMARY OF INVENTION

In accordance with one aspect, the invention provides a crystalline form of Cetirizine monohydrochloride. Preferably, the crystalline form of Cetirizine monohydrochloride has an X-ray diffraction pattern, expressed in terms of 2θ angles and obtained with a diffractometer equipped with a copper K X-radiation source, wherein said X-ray powder diffraction pattern includes five or more peaks selected from the group consisting of peaks with 2 theta angles of 12.97±0.09, 22.94±0.09, 20.41±0.09, 17.35±0.09, 19.15±0.09, 8.75±0.09, 14.19±0.09, 17.01±0.09, 28.28±0.09, 21.61±0.09 and 9.84±0.09 degrees. Various embodiments and variants are also provided.

In accordance with another aspect, the invention provides a composition containing solid cetirizine monohydrochloride. Preferably, at least 80% by weight of the solid cetirizine monohydrochloride in the composition is in a crystalline form. The composition may further include at least one additional form of solid cetirizine different from cetirizine monohydrochloride. Suitable examples of additional forms of solid cetirizine are cetirizine free species and cetirizine dihydrochloride. In one embodiment of this aspect of the invention, there is contemplated a composition in which the at least one different form of solid cetirizine is cetirizine dihydrochloride, which is present in the amount of from about 80% to about 99.5% by weight with respect to the combined weight of all solid forms of cetirizine in the composition. In this embodiment, the crystalline form of cetirizine monohydrochloride is present in the amount of from about 0.1% to about 5% by weight with respect to the combined weight of all solid forms of cetirizine in the composition. In one particularly preferred embodiment of this aspect of the invention, the composition is a bulk solid suitable for pharmaceutical formulation in which cetirizine dihydrochloride is the major component and crystalline cetirizine monohydrochloride is the minor component. Various embodiments and variants are also provided.

The invention also relates to a process for preparing the crystalline form of Cetirizine monohydrochloride and to a pharmaceutical composition that includes the crystalline form of Cetirizine monohydrochloride and one or more pharmaceutically acceptable carriers or diluents. The pharmaceutical composition may also include one or more additional active ingredients. Preferably, the pharmaceutical composition is in a solid dosage form for oral administration, such as a tablet.

The invention also relates to a method of preventing or treating allergic syndromes, comprising administering to a patient in need of such treatment an effective amount of crystalline form of Cetirizine monohydrochloride. A method of making cetirizine dihydrochloride from crystalline, solid cetirizine monohydrochloride is also contemplated.

### DESCRIPTION OF THE ACCOMPANYING DRAWINGS

FIG. 1 is an X-ray powder diffractogram of the crystalline form of Cetirizine monohydrochloride.

FIG. 2 is an infrared spectrum of the crystalline form of Cetirizine monohydrochloride.

FIG. 3 is a differential scanning calorimetry thermogram of the crystalline form of Cetirizine monohydrochloride.

### DETAILED DESCRIPTION OF THE INVENTION

Unless defined otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

For purposes of the present invention, the following terms are defined below.

A “compound” is a chemical substance that includes molecules of the same chemical structure.

37 Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes, but is not limited to, a powder, a suspension, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction, which produces the indicated and/or the desired product, may not necessarily result directly from the combination of two reagents, which were initially added, i.e., there may be one or more intermediates which are produced in the mixture which ultimately leads to the formation of the indicated and/or the desired product. Also, the term “isolating” is used to indicate separation of the compound being isolated regardless of the purity of the isolated compound from any unwanted substance which presents with the compound as a mixture. Thus, degree of the purity of the isolated or separated compound does not affect the status of “isolating”.

The term “substantially free of” in reference to a composition, as used herein, means that said substance cannot be detected in the composition by methods known to those skilled in the art at the time of the filing of this application.

Cetirizine monohydrochloride has the structure,

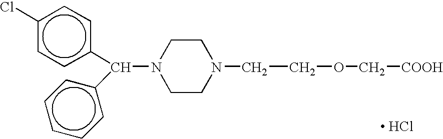

The preparation of Cetirizine generally is known in the art. For example, the processes for the preparation of Cetirizine and its dihysrochloride salt are disclosed U.S. Pat. No. 4,525,358, of which entire content is incorporated by reference herein. The disclosed process involves hydrolysis of the methyl ester of Cetirizine using ethanolic potassium hydroxide to afford potassium salt of Cetirizine. The solution containing the potassium salt is then acidified with hydrochloric acid. U.S. Pat. No. 6,255,487, incorporated by reference, discloses a process for the preparation of Cetirizine dihydrochloride via condensation of (4-chloro phenyl)phenyl methyl chloride and potassium 2-(1-piperazinyl)ethoxyacetate in acetonitrile, followed by acidification in acetone medium with concentrated hydrochloric acid.

It is known that polymorphic forms of the same drug may have substantial differences in certain pharmaceutically important properties such as dissolution characteristics and bioavailability as well as stability of the drug. Furthermore, difference crystalline form may have different particle size, hardness and glass transition temperature. Thus, one crystalline form may provide significant advantages over other crystalline forms of the same drug in solid dosage form manufacture process such as accurate measurement of the active ingredients, easier filtration, or improved stability during granulation or storage. Furthermore, a particular process suitable for one crystalline form may also provide drug manufacturers several advantages such as economically or environmentally suitable solvents or process, or higher purity or yield of the desired product.

Thus, according to one aspect, the present invention provides a novel crystalline form of Cetirizine monohydrochloride. The crystalline form of Cetirizine monohydrochloride of the present invention may be prepared from unpurified Cetirizine monohydrochloride or from Cetirizine or its other salts. For example, Cetirizine dihydrochloride may be deionized in a basic aqueous solvent and reacted with HCl again to form Cetirizine monohydrochloride. The basic aqueous solvent could be just water, which is basified with a typical inorganic base such as sodium hydroxide, potassium hydroxide, sodium carbonate, potassium carbonate or its bicarbonates, etc to a pH between about 8 and about 14 before or after adding Cetirizine dihydrochloride into the water. After basified, the aqueous solution formed therefrom is acidified with HCl to a pH between about 2 and 4. Then, the aqueous solution is subjected to extraction with a water immiscible solvent such as dichloromethane, chloroform, ethyl acetate, or mixtures thereof. After the solvent of the organic layer is removed by a conventional method, the residue obtained thereby is dissolved in a ketone solvent such as acetone, ethyl methyl ketone, methyl isobutyl ketone or mixtures thereof. Stirring the resulted ketone solution at a room temperature gives separation of a solid mass, which can be isolated by a conventional method to give the crystalline form of Cetirizine monohydrochloride.

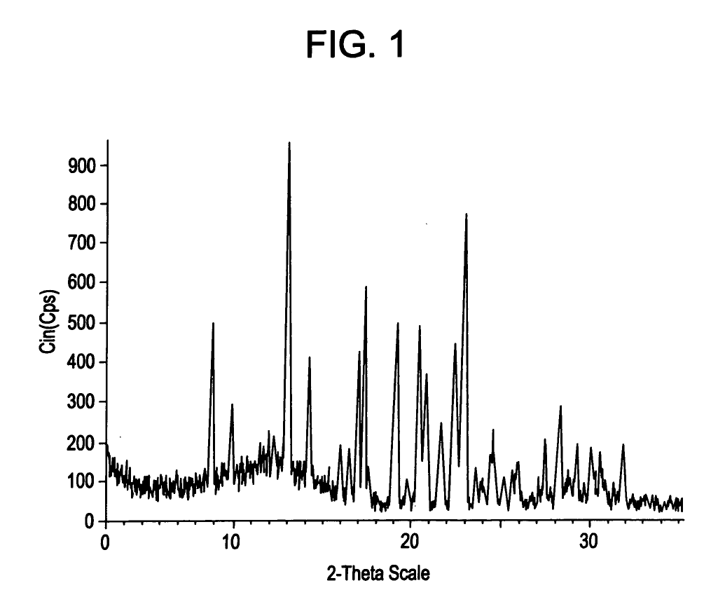

The crystalline form of Cetirizine monohydrochloride produced by this process was characterized by an X-ray powder diffraction pattern, as for example shown in FIG. 1, and the characteristic 2 theta values (in degrees) and intensities of the identified peaks in the X-ray diffractograms are shown in Table 1:

TABLE 1

| 2-Theta | Intensity, |
|---|---|
| Value (°) | I/Iₒ (%) |
| 12.968 | 100.0 |
| 22.941 | 98.1 |
| 20.405 | 59.1 |
| 17.348 | 57.5 |
| 19.148 | 54.3 |
| 8.749 | 41.5 |
| 14.189 | 39.4 |
| 17.01 | 37.9 |
| 28.28 | 27.6 |
| 21.610 | 22.1 |
| 9.842 | 21.4 |

The X-ray diffractogram was measured on a Bruker Axs, D8 Advance Powder X-ray Diffractometer with Cu K alpha-1 radiation source.

It should be kept in mind that slight variations in the observed 2 theta angles values are expected based on the specific diffractometer employed, the analyst and the sample preparation technique. More variation is expected for the relative peak intensities, which is largely affected by the particle size of the sample. Thus, identification of the exact crystalline form of a compound should be based primarily on observed 2 theta angles with lesser importance attributed to relative peak intensities. The peaks reported herein are listed in order of their peak intensities. Thus, the first listed peak has stronger intensity than the second listed peak in the pattern. The 2 theta diffraction angles and corresponding d-spacing values account for positions of various peaks in the X-ray powder diffraction pattern. D-spacing values are calculated with observed 2 theta angles and copper K(α1) wavelength using the Bragg equation well known to those of skill in the art.

Thus, some margin of error may be present in each of the 2 theta angle assignments reported herein. The assigned margin of error in the 2 theta angles for the crystalline form of Cetirizine monohydrochloride is approximately ±0.009 for each of the peak assignments. In view of the assigned margin of error, in a preferred variant, the crystalline form of Cetirizine monohydrochloride may be characterized by an X-ray diffraction pattern, expressed in terms of 2 theta angles, that includes four or more peaks selected from the group consisting of 12.97±0.09, 22.94±0.09, 20.41±0.09, 17.35±0.09, 19.15±0.09, 8.75±0.09, 14.19±0.09, 17.01±0.09, 28.28±0.09, 21.61±0.0 and 9.84±0.09.

Since some margin of error is possible in the assignment of 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified crystalline form of Cetirizine monohydrochloride over FIG. 1 and readily determine whether the X-ray diffraction pattern of the unidentified form is substantially the same as the X-ray powder diffraction pattern of the crystalline form of this invention. If the X-ray powder diffraction pattern is substantially the same as FIG. 1, the previously unknown crystalline form of Cetirizine monohydrochloride can be readily and accurately identified as the crystalline form of this invention.

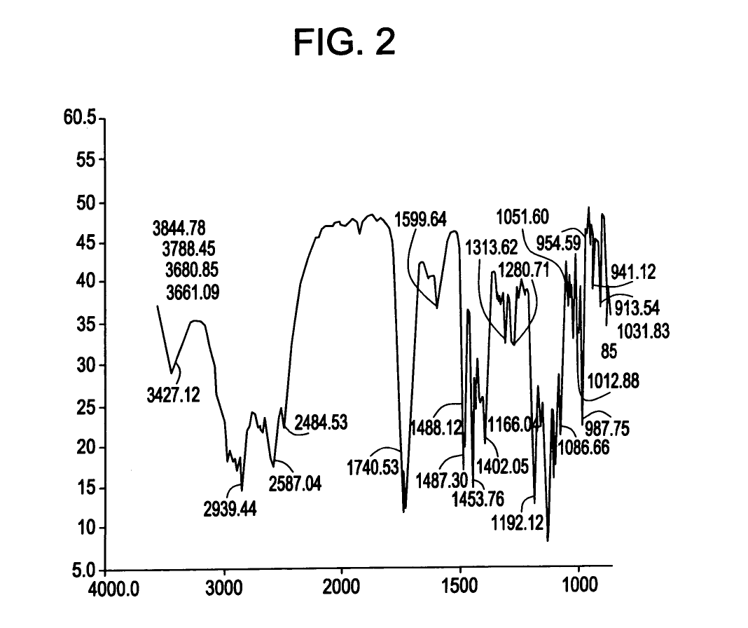

The crystalline forms of Cetirizine monohydrochloride was also characterized by Infrared spectroscopy (IR) as shown respectively in FIG. 2. In addition to the finger print region of the IR spectrum, the peak locations of several distinctive peaks may help one of skill in the art to identify the crystalline form of the present invention. These peaks include absorption bands at about 3427 cm⁻¹, about 2839 cm⁻¹, about 2587 cm⁻¹, about 1741 cm⁻¹, and about 1600 cm⁻¹.

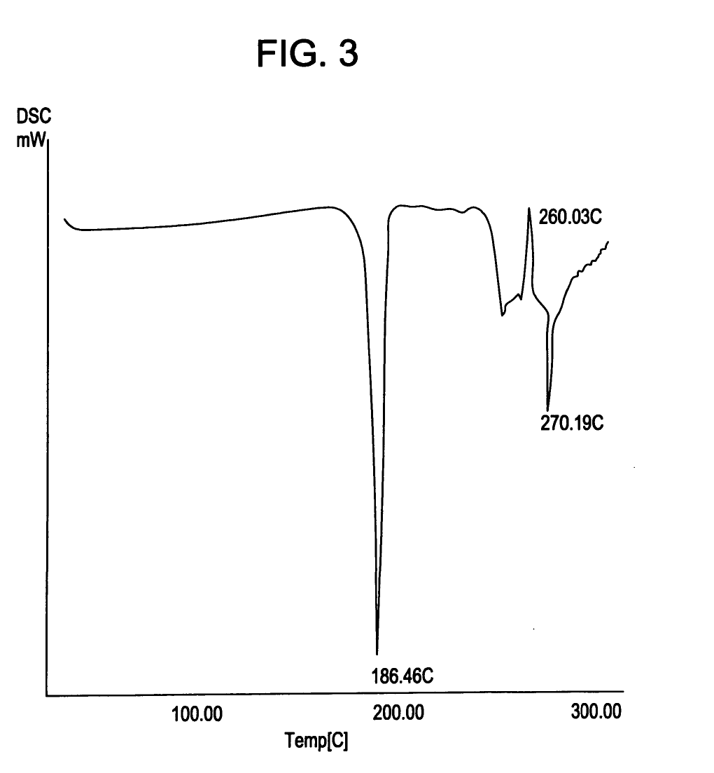

The Differential scanning calorimetry (DSC) thermogram of crystalline form of Cetirizine monohydrochloride obtained by the inventors is shown in FIG. 3. It exhibits a significant endo-exo pattern with identified peaks around 186° C. and 260° C. The DSC spectrum was measured on a Perkin Elmer. The melting point of the crystalline Form Z was also measured by the capillary method and was determined to be 183-189° C.

The invention also relates to a composition containing solid Cetirizine monohydrochloride of which at least 80%, by total weight of the solid Cetirizine monohydrochloride in the composition, is the crystalline form. In the more preferred form of this composition, the solid Cetirizine monohydrochloride is suitable for use as active ingredient in formulating pharmaceutical products. In an embodiment of the invention, the composition may comprise at least 90% of crystalline form of Cetirizine monohydrochloride with respect to total weight of the solid Cetirizine monohydrochloride in the composition. In another embodiment of the invention, the composition may comprise at least 95% of crystalline form of Cetirizine monohydrochloride with respect to total weight of the solid Cetirizine monohydrochloride in the composition. In yet another embodiment of the invention, the composition is substantially free of any forms of Cetirizine monohydrochloride other than its crystalline form.

In one particular embodiment, there is provided a composition which contains the crystalline cetirizine monohydrochloride and at least one different form of solid cetirizine, such as cetirizine free species or cetirizine dihydrochloride. In one variant, the additional solid form of cetirizine is cetirizine dihydrochloride which is present in the amount of from about 80% to about 99.5% by weight with respect to the combined weight of all solid forms of cetirizine in the composition. In this variant, the crystalline form of cetirizine monohydrochloride is present in the amount of from about 0.1% to about 5% by weight with respect to the combined weight of all solid forms of cetirizine in the composition. In one particularly preferred embodiment of this aspect of the invention, the composition is a bulk solid suitable for pharmaceutical formulation in which cetirizine dihydrochloride is the major component and crystalline cetirizine monohydrochloride is the minor component. An example of suitable form of solid cetirizine dihydrochloride is the crystalline Form I thereof, XRD pattern of which, obtained on a Bruker Axs, D8 Advance Powder X-ray Diffractometer with Cu K alpha-1 Radiation source, is exemplified in Table 2:

| 2-Theta | Intensity, |
|---|---|
| Value (°) | I/Iₒ (%) |
| 18.637 | 100.0 |
| 18.244 | 81.1 |
| 25.115 | 78.8 |
| 14.423 | 47.9 |
| 17.328 | 35.9 |
| 8.007 | 28.0 |
| 20.388 | 27.8 |
| 24.143 | 25.8 |
| 7.099 | 25.4 |
| 14.731 | 22.5 |
| 23.432 | 20.7 |
| 12.966 | 20.9 |
| 22.949 | 17.8 |
| 26.109 | 16.5 |
| 29.204 | 11.3 |
| 26.706 | 10.7 |
| 8.756 | 9.9 |
| 19.965 | 9.0 |
| 15.923 | 8.8 |
| 13.526 | 8.5 |
| 33.796 | 7.3 |
| 34.342 | 7.3 |
| 12.592 | 7.0 |
| 35.044 | 6.0 |
| 16.676 | 5.1 |
| 43.147 | 4.8 |

The preparation of the crystalline Form I of cetirizine dihydrochloride is described in co-pending and co-assigned Indian Patent Application No. 425/MAS/2002 and International Application No. PCT/US03/17672, both of which are incorporated by reference both for the specific reason described and for entirety of disclosure.

X-ray diffraction provides a convenient and practical means for quantitative determination of the relative amounts of crystalline and/or amorphous forms in a solid mixture. X-ray diffraction is adaptable to quantitative applications because the intensities of the diffraction peaks of a given compound in a mixture are proportional to the fraction of the corresponding powder in the mixture. The percent composition of crystalline Cetirizine monohydrochloride in an unknown composition can be determined. Preferably, the measurements are made on solid powder Cetirizine monohydrochloride. The X-ray powder diffraction patterns of an unknown composition can be compared to known quantitative standards containing the pure crystalline form of Cetirizine monohydrochloride to identify the percent ratio of a particular crystalline form. This is done by comparing the relative intensities of the peaks from the diffraction pattern of the unknown solid powder composition with a calibration curve derived from the X-ray diffraction patterns of pure known samples. The curve can be calibrated based on the X-ray powder diffraction pattern for the strongest peak from a pure sample of the crystalline form of Cetirizine monohydrochloride. The calibration curve may be created in a manner known to those of skill in the art. For example, five or more artificial mixtures of crystalline forms of Cetirizine monohydrochloride, at different amounts, may be prepared. In a non-limiting example, such mixtures may contain, 2%, 5%, 7%, 8%, and 10% of the crystalline Cetirizine monohydrochloride. Then, X-ray diffraction patterns are obtained for each artificial mixture using standard X-ray diffraction techniques. Slight variations in peak positions, if any, may be accounted for by adjusting the location of the peak to be measured. The intensities of the selected characteristic peak(s) for each of the artificial mixtures are then plotted against the known weight percentages of the crystalline form. The resulting plot is a calibration curve that allows determination of the amount of the crystalline form of Cetirizine monohydrochloride in an unknown sample. For the unknown mixture of the crystalline and amorphous forms of Cetirizine monohydrochloride, the intensities of the selected characteristic peak(s) in the mixture, relative to an intensity of this peak in a calibration mixture, may be used to determine the percentage of the given crystalline form in the composition, with the remainder determined to be the amorphous material.

Pharmaceutical compositions comprising crystalline form of Cetirizine monohydrochloride can be formulated with one or more pharmaceutically acceptable carriers, also known as excipients, which ordinarily lack pharmaceutical activity, but have various useful properties which may, for example, enhance the stability, sterility, bioavailability, and ease of formulation of a pharmaceutical composition. These carriers are pharmaceutically acceptable, meaning that they are not harmful to humans or animals when taken appropriately and are compatible with the other ingredients in a given formulation. The carriers may be solid, semi-solid, or liquid, and may be formulated with the compound in bulk. The resulting mixture may be manufactured in the form of a unit-dose formulation (i.e., a physically discrete unit containing a specific amount of active ingredient) such as a tablet or capsule.

The pharmaceutical compositions may include, in addition to a compound of this invention, one or more active pharmaceutical compounds. For example, U.S. patent Publication No. 2002/0012700, incorporated by reference, discloses a combination dosage form comprising Cetirizine and pseudoephedrine. Similarly, U.S. patent Publication No. 2002/0099058, incorporated by reference, discloses pharmaceutical compositions containing Cetirizine and a leukotriene inhibitor and its pharmaceutically acceptable salts such as zileuton. Also U.S. Pat. No. 4,829,064, incorporated by reference, discloses compositions useful for treating cold symptoms comprising Cetirizine and an analgesic. Thus, the crystalline form of the present invention may also be combined with pseudoephedrine, a leukotriene inhibitor or an analgesic to utilize the advantages of the present invention.

Generally, the pharmaceutical compositions of the invention may be prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed.

A preferred oral solid preparation is a tablet. A tablet may be prepared by direct compression, wet granulation, or molding, of the active ingredient(s) with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine. A mixture of the powdered compound moistened with an inert liquid diluent is suitable in the case of oral solid dosage forms (e.g., powders, capsules, and tablets). If desired, tablets may be coated by standard techniques. The compounds of this invention may be formulated into typical disintegrating tablets, or into controlled or extended release dosage forms.

The pharmaceutical compositions of the invention are contemplated in various formulations suitable for various modes of administration, including but not limited to inhalation, oral, rectal, parenteral (including subcutaneous, intradermal, intramuscular, intravenous), implantable, intravaginal and transdermal administration. The most suitable route of administration in any given case depends on the duration of the subject's condition, the length of treatment desired, the nature and severity of the condition being treated, and the particular formulation that is being used. The formulations may be in bulk or in unit dosage form.

The amount of active ingredient included in a unit dosage form depends on the type of formulation that is formulated. A pharmaceutical composition of the invention will generally comprise about 0.1% by weight to about 99% by weight of active ingredient, preferably about 1% by weight to 50% by weight for oral administration and about 0.2% by weight to about 20% by weight for parenteral administration.

Formulations suitable for oral administration include capsules (hard and soft), cachets, lozenges, syrups, suppositories, and tablets, each containing a pre-determined amount of the active compound; as a powder or granules; as a solution or a suspension in an aqueous or non-aqueous liquid; or as an oil-in-water or water-in-oil emulsion. Such formulations may be prepared by any suitable method of pharmacy that includes the step of bringing into association the active compound and a suitable carrier or carriers. For liquid oral formulations, a preferable amount is from about 2% by weight to about 20% by weight. Suitable carriers include but are not limited to fillers, binders, lubricants, inert diluents, surface active/dispersing agents, flavorants, antioxidants, bulking and granulating agents, adsorbants, preservatives, emulsifiers, suspending and wetting agents, glidants, disintegrants, buffers and pH-adjusting agents, and colorants. Examples of carriers include celluloses, modified celluloses, cyclodextrins, starches, oils, polyols, sugar alcohols and sugars, and others. For liquid formulations sugar, sugar alcohols, ethanol, water, glycerol, and poyalkylene glycols are particularly suitable, and may also be used in solid formulations. Cyclodextrins may be particularly useful for increasing bioavailability. Formulations for oral administration may optionally include enteric coatings known in the art to prevent degradation of the formulation in the stomach and provide release of the drug in the small intestine.

Formulations suitable for buccal or sub-lingual administration include lozenges comprising the active compound in a flavored base, usually sucrose and acacia or tragacanth, although other agents are also suitable, and pastilles comprising the compound in an inert base such as gelatin and glycerin or sucrose and acacia.

Formulations suitable for rectal administration are preferably presented as unit dose suppositories. These may be prepared by admixing the active compound with one or more conventional solid carriers, e.g., cocoa butter, and then shaping the resulting mixture.

In another aspect, the invention also provides methods of preventing or treating a treatment of allergic syndromes, such as chronic and acute allergic rhinitis including seasonal and perennial allergic rhinits, allergic conjunctivitis, pruritus, urticaria, and the like.

The effective amount (i.e., dosage) of active compound for treatment will vary depending on the route of administration, the condition being treated, its severity, and duration, and the state and age of the subject. A skilled physician will monitor the progress of the subject and will adjust the dosage accordingly, depending on whether the goal is to eliminate, alleviate, or prevent a given condition. Generally, the dosage should be considered in proportion to the subject's weight. The daily dose of particular formulations of active compound may be divided among one or several unit dose administrations. For example therapeutic administration about fifteen to thirty minutes before main meals is preferable (i.e. three times daily), although administration of the active compounds may be carried out prophylactically, and may be maintained for prolonged periods of time. One skilled in the art will take such factors into account when determining dosage. Unit dosage of active ingredient may range preferably from about 1 mg to about 100 mg, more preferably from about 10 mg to about 50 mg.

The invention is further described by reference to the following examples which set forth in detail the preparation of compounds and compositions of the present invention, as well as their utility. It will be apparent to those skilled in the art, that many modifications, both to materials, and methods, may be practiced without departing from the purpose and interest of this invention. The examples that follow are not intended to limit the scope of the invention as described hereinabove or as claimed below.

### REFERENCE EXAMPLE 1

### Preparation of Cetirizine

[2-[4-[(4-Chlorophenyl)-phenyl methyl]-1-piperazinyl]ethoxy]acetamide (50 grams) in 6.5% w/v aqueous sodium hydroxide (200 ml) was refluxed till the reaction was substantially completes. Then the reaction mixture was cooled, further diluted with water (300 ml) accompanied by adjusting the pH of the reaction solution around 9.0 with concentrated hydrochloric acid, washed the resulted reaction mass with ethyl acetate. The pH of the separated aqueous layer was further adjusted to 4.0 with concentrated hydrochloric acid and extracted with dichloromethane. Then the combined dichloromethane layer was evaporated under vacuum to get the required Cetirizine base. (43.0 grams).

### REFERENCE EXAMPLE 2

### Preparation of Cetirizine dihydrochloride

Cetirizine was suspended in 150 ml of water, the pH of which was adjusted to 08 by addition of concentrated hydrochloric acid. The aqueous solution was concentrated on a rotary evalporator, and the residue as then diluted by addition of 75 ml of 2-butanone, which results in crystallization of Cetirizine dihydrochloride. The crystals was isolated by filtration and dried to yield the desired Cetirizine dihydrochloride.

### EXAMPLE 1

### Preparation of Crystalline form of Cetirizine monohydrochloride from Cetirizine dihydrochloride

Cetirizine dihydrochloride (150 grams) was dissolved in water (1500 ml). The pH of the solution was adjusted to pH 12 using sodium hydroxide. Subsequently, the pH of the solution was adjusted to 2-3 with hydrochloric acid and the product was extracted with dichloromethane (2×450 ml). The organic layer was washed with water (2×150 ml), dried with sodium sulfate and evaporated under reduced pressure. The residue was washed with acetone (300 ml), acetone evaporated, then washed again in acetone (900 ml) and stirred at 40-45° C. until separation. The residue was filtered, washed with acetone (150 ml) and dried at 50-60° C. yielding 87.5 g of the Cetirizine monohydrochloride. Melting point 183-189° C.

### EXAMPLE 2

### Preparation of cetirizine dihydrochloride from Solid Crystalline cetirizine monohydrochloride

A solid powder of cetirizine monohydrochloride (10.0 grams) is stirred in ethyl acetate (100 ml) at a temperature of 25-35° C. for 10-15 min. Isopropanolic hydrochloric acid (15 ml) is added till the pH of reaction mass becomes 2.0. The reaction mass is stirred for 1-2 hours to separate the solid. The separated solid is filtered, washed with ethyl acetate (20 ml), followed by hexane (10 ml) and on subsequent drying at a temperature of 80-100° C. to a constant weight provides solid of cetirizine dihydrochloride.

### EXAMPLE 3

### Soluble Granules Containing Crystalline Cetirizine Monohydrochloride

Soluble granules containing crystalline Cetirizine monohydrochloride may have the following content:

| Ingredient | Content (mg) |
|---|---|
| Crystalline Cetirizine monohydrochloride | 10 |
| Calcium carbonate | 800 |
| Citric acid | 900 |
| Avicel | 40 |
| Mannitol | 625 |
| Maltodextrin | 15 |
| Aspartame | 3 |
| Aroma | 20 |

### EXAMPLE 4

### Dispersible Tablet Containing Crystalline cetirizine monohydrochloride

Dispersible tablet containing crystalline Cetirizine monohydrochloride may have the following content:

| Ingredient | Content (mg) |
|---|---|
| Crystalline Cetirizine monohydrochloride | 10 |
| Calcium carbonate | 500 |
| Polyvinylpyrrolidone | 17 |
| Avicel | 15 |
| Mannitol | 400 |
| Maltodextrin | 15 |
| Aspartame | 3 |
| Aroma | 20 |

Unless stated to the contrary, words and phrases such as “including,” “containing,” “comprising,” “having”, “for example”, “i.e.”, “in particular” and the like, means “including without limitation” and shall not be construed to limit any general statement that it follows to the specific or similar items or matters immediately following it. Except where the context indicates to the contrary, all exemplary values are intended to be used for purposes of illustration. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

1. A crystalline form of cetirizine monohydrochloride.

2. The crystalline form of cetirizine monohydrochloride of claim 1 having an X-ray diffraction pattern, expressed in terms of 2θ angles and obtained with a diffractometer equipped with a copper K X-radiation source, wherein said X-ray powder diffraction pattern includes five or more peaks selected from the group consisting of peaks with 2 theta angles of 12.97±0.09, 22.94±0.09, 20.41±0.09, 17.35±0.09, 19.15±0.09, 8.75±0.09, 14.19±0.09, 17.01±0.09, 28.28±0.09, 21.61±0.09 and 9.84±0.09 degrees.

3. The crystalline form of cetirizine monohydrochloride of claim 1, wherein said X-ray diffraction pattern includes peaks with 2 theta angles of about 12.968, 22.941, 20.405, 17.348, 19.148, 8.749, 14.189, 17.01, 28.28, 21.610, and 9.842.

4. The crystalline form of cetirizine monohydrochloride of claim 1 having substantially the same X-ray diffraction pattern as shown in FIG. 1.

5. The crystalline form of cetirizine monohydrochloride of claim 1 having an infrared absorption spectrum comprising absorption bands at about 3427 cm⁻¹, about 2839 cm⁻¹, about 2587 cm⁻¹, about 1741 cm⁻¹, and about 1600 cm⁻¹.

6. The crystalline form of cetirizine monohydrochloride of claim 1 having substantially the same infrared spectrum as shown in FIG. 2.

7. The crystalline form of Cetirizine monohydrochloride of claim 1 having a differential scanning calorimetry thermogram, which exhibits an endotherm peak at about 186° C.

8. The crystalline form of cetirizine monohydrochloride of claim 1 having substantially the same differential scanning calorimetry thermogram as shown in FIG. 3.

9. A composition comprising cetirizine monohydrochloride as a solid, wherein at least 80% by weight of said solid cetirizine monohydrochloride is in a crystalline form.

10. The composition of claim 9, further comprising at least one additional form of solid cetirizine different from cetirizine monohydrochloride, said at least one different form of solid cetirizine being selected from the group consisting of cetirizine free species and cetirizine dihydrochloride.

11. The composition of claim 10, wherein said at least one different form of solid cetirizine is cetirizine dihydrochloride being present in the amount of from about 80% to about 99.5% by weight with respect to the combined weight of all solid forms of cetirizine in the composition.

12. The composition of claim 11, wherein said crystalline form of cetirizine monohydrochloride is present in the amount of from about 0.1% to about 5% by weight with respect to the combined weight of all solid forms of cetirizine in the composition.

13. The composition of claim 9, which is in the form of bulk powder suitable for use as an active ingredient in pharmaceutical formulations.

14. The composition of claim 9, where said crystalline cetirizine monohydrochloride has an X-ray diffraction pattern, expressed in terms of 2θ angles and obtained with a diffractometer equipped with a copper K X-radiation source, wherein said X-ray powder diffraction pattern includes five or more peaks selected from the group consisting of peaks with 2 theta angles of 12.97±0.09, 22.94±0.09, 20.41±0.09, 17.35±0.09, 19.15±0.09, 8.75±0.09, 14,19±0.09, 17.01±0.09, 28.28±0.09, 21.61±and 9.84±0.09 degrees.

15. The composition of claim 9, wherein at least 90% by weight of said solid cetirizine monohydrochloride is in said crystalline form.

16. The composition of claim 9, wherein at least 95% by weight of said solid cetirizine monohydrochloride is in said crystalline form.

17. The composition of claim 9, wherein at least 99% by weight of said solid cetirizine monohydrochloride is in said crystalline form.

18. A pharmaceutical composition, which comprises a pharmaceutically effective amount of a crystalline form of cetirizine monohydrochloride and one or more pharmaceutically acceptable carriers or diluents.

19. The pharmaceutical composition of claim 18, wherein said crystalline form of cetirizine monohydrochloride has an X-ray diffraction pattern expressed in terms 2 theta angles and obtained with a copper K X-radiation source, wherein said X-ray powder diffraction pattern includes five or more peaks selected from the group consisting of peaks with 2 theta angles of 12.97±0.09, 22.94±0.09, 20.41±0.09, 17.35±0.09, 19.15±0.09, 8.75±0.09, 14,19±0.09, 17.01±0.09, 28.28±0.09, 21.61±0.09 and 9.84±0.09 degrees.

20. The pharmaceutical composition of claim 18, which is a solid dosage form for oral administration.

21. The pharmaceutical composition of claim 20, wherein said solid dosage form is a tablet.

22. The pharmaceutical composition of claim 18, which further comprises of one or more additional active ingredients.

23. The pharmaceutical composition of claim 22, wherein said additional active ingredient is pseudoephedrine.

24. The pharmaceutical composition of claim 22, wherein said additional active ingredient is a leukotriene inhibitor.

25. The pharmaceutical composition of claim 22, wherein said additional active ingredient is an analgesic.

26. A process for preparation of a crystalline form of cetirizine monohydrochloride, said process comprising:
a. providing a solid residue of crude cetirizine monohydrochloride;
b. contacting said crude residue with a ketone solvent to cause separation of a solid mass; and
c. isolating said solid mass thereby obtaining said crystalline form of cetirizine monohydrochloride.

27. The process of claim 26, further comprising providing an aqueous solution of cetirizine or salts thereof, adjusting the pH of said aqueous solution to between about 2 and about 4 with hydrochloric acid, extracting the acidified aqueous solution with a water immiscible organic solvent, and removing said water immiscible solvent to form said solid residue of cetirizine monohydrochloride.

28. The process of claim 27, wherein said water immiscible organic solvent is selected from the group consisting of dichloromethane, chloroform, dichloroethane, ethyl acetate, and mixtures thereof.

29. The process of claim 27, wherein said water immiscible solvent is dichloromethane.

30. The process of claim 26, wherein said ketone solvent is selected from a group consisting of acetone, ethyl methyl ketone, methyl isobutyl ketone, and mixtures thereof.

31. The process of claim 26, wherein said ketone solvent is acetone.

32. The cetirizine monohydrochloride produced in accordance with the process of claim 26.

33. A method of treating allergic syndromes, which comprises administering a mammal in need thereof an effective amount of the compound of claim 1.

34. The method of claim 33, wherein said mammal is a human.

35. A process of making cetirizine dihydrochloride, which comprises:
a) providing a solid which is a cetirizine monohydrochloride in an amorphous form;
b) contacting said solid cetirizine monohydrochloride with a liquid phase containing water;
c) adding one or more equivalents of hydrochloric acid to said liquid phase so that said cetirizine monohydrochloride is converted to said cetirizine dihydrochloride.

36. The process of claim 35, further comprising dissolving said solid cetirizine monohydrochloride in an organic solvent prior to said contacting step.

37. The process of claim 35, wherein said organic solvent is selected from the group consisting of dichloromethane, chloroform, dichloroethane, ethyl acetate, methyl acetate and mixtures thereof.

38. The process of claim 35, wherein said hydrochloric acid is added in an alcoholic solution.
